(19) United States
(12) Patent Application Publication
(10) Pub. No.: US 2025/0328007 A1
(43) Pub. Date: Oct. 23, 2025

(54) MICROSCOPE DISINFECTING SYSTEM

(71) Applicant: eyeMobil Innovations, LLC, Niles, MI (US)

(72) Inventor: Sam Powdrill, Wilmore, KY (US)

(21) Appl. No.: 19/184,283

(22) Filed: Apr. 21, 2025

Related U.S. Application Data
(60) Provisional application No. 63/637,099, filed on Apr. 22, 2024.

(51) Int. Cl. G02B 27/00; A61L 2/10

(52) U.S. Cl. CPC G02B 27/0006; A61L 2/10

(57) ABSTRACT

The present disclosure relates to a microscope disinfecting system configured to disinfect the optical elements of a microscope. The microscope disinfecting system includes one or more adapters, a substantially Y-shaped connector, and an electric connector. Each adapter includes an inner cavity dimensioned to receive an ocular lens of a microscope. The inner cavity includes a light source capable of producing ultraviolet C (UV-C) light. The multiple sizes of interchangeable adapter rings of the adapter allow the microscope disinfecting system to disinfect ocular lenses of any size ranging between 30 mm and 50 mm.

### CROSS-REFERENCE TO RELATED APPLICATIONS

The present application hereby claims priority to and incorporates by reference the entirety of the disclosures of the provisional application No. 63/637,099 entitled “MICROSCOPE DISINFECTING SYSTEM”, filed on Apr. 22, 2024.

### FIELD OF THE INVENTION

The present invention relates to the field of microscopes, and more particularly, the present invention relates to a microscope disinfecting system that involves the use of light to kill bacteria, fungi, or other microorganisms present on the optical elements of the microscope.

### BACKGROUND

In uncontrolled climatic conditions, it is common for fungi to grow on the surfaces of optical components. Airborne fungal spores settle on optical surfaces and develop into organisms that digest organic material, such as oils from fingerprints or lens coatings, producing hydrofluoric acid as a waste product. This acid in turn destroys any lens coatings and permanently etches the glass. Thus, the resultant image produced in the microscope is negatively affected (obscured) due to the biological growth of fungi and bacteria.

Removing fungus from the lenses of a microscope can be difficult and may not yield suitable results since the damage is often permanent. Killing and removing the fungus and cleaning the optical surface using chemicals may prolong the useful life of the instrument, however, only if it can still provide an acceptable image. The problem of fungal growth on the optical elements of a surgical viewing system can occur in non-climate-controlled conditions where humidity and temperature are appropriate for fungal growth.

Commonly used surface disinfectants for the removal of microorganisms such as but not limited to bacteria, fungi, etc., contain alcohols, aldehydes, chlorine compounds, phenols, and peroxides. The effectiveness of disinfection depends on the concentration of the disinfectant, the time of contact with the material, and the type of microbes present. In addition to the disinfectant, cotton swabs, lens cleaning paper, and latex gloves are also used. Further, chemicals such as a 50/50 mix of hydrogen peroxide (H202) and ammonia (NH3) can be used for killing and removing the fungus from the optical surfaces of the microscope. However, these methods are inefficient in use and often require repurchasing several times during the use of the microscope, which in turn becomes a costlier affair and causes inconvenience for the users.

Further, in the past, antifungal tablets that contain antifungal compounds have been used inside a microscope to prevent fungal growth. However, such tablets have a limited half-life and need to be replaced regularly, and are not easily available. Further, such tablets are not widely available for use with all types of microscopes and have limited use with the microscopes from specific vendors.

Additionally, many microscopes come with some level of antifungal coating at the time of manufacture, but such coatings don't fully protect the lenses over time. For instance, US20060262390A1 discloses a microscope wherein at least a surface portion of a microscope, for example, the stand, the stage, the eyepiece, or the several operating elements, is provided with an antimicrobial outer surface. In an embodiment, an antimicrobial outer surface can be provided on the surfaces of a microscope that are not typically contacted by a microscope user during regular use; for example, a microbial surface may be provided on internal surfaces to prevent the growth of microbes, fungi, or other micro-organisms, etc. that may affect microscope performance, e.g. lens tubes, etc.

US20060238857A1 discloses a surgical microscope system comprises a stand with a carrier arm, an optical unit having a microscope body, an objective, and at least one eyepiece tube, and at least one input unit having a display and a touch-based operating panel, the touch-based operating panel being antimicrobial, e.g. as a result of the addition of silver, copper, or other chemical substances having antimicrobial properties.

US20060034423A1 discloses a surgical microscope whose surface is at least partly provided with an incorporated bactericidal and/or fungicidal material. This surface part (A-D) comprises at least one carrier material with an incorporated bactericidal and/or fungicidal material dispersed therein in the form of nanoparticles having a size of <20 nm. Accordingly, it is intended that at least one carrier material with incorporated bactericidal and/or fungicidal material dispersed therein in the form of nanoparticles having a size of <20 nm be used for at least one part-surface (A-D) of a surgical microscope.

The existing solutions related to microscope disinfection are ineffective, significantly add on the cost of usage of the microscope, are difficult to manufacture, tend to damage the microscope, and are inefficient in use as they fail to provide a microscope disinfecting system that can effectively and efficiently kill the bacteria and fungus present on the optical elements of the microscope. Thus, there is a need for a more efficient solution that would solve the aforementioned problem of microorganisms building up on the microscope by providing a microscope disinfecting system that involves the use of light to kill bacteria, fungi, or similar microorganisms present on the optical elements of the microscope.

### SUMMARY

An object of the present invention is to provide a microscope disinfecting system that can effectively and efficiently kill the bacteria, fungus, and similar microorganisms present on the optical elements of the microscope.

An object of the present invention is to provide a microscope disinfecting system that can be used several times during the lifetime of the microscope.

An object of the present invention is to provide a microscope disinfecting system that can be used to disinfect various types of microscopes of different sizes made by different vendors.

An object of the present invention is to provide a microscope disinfecting system that does not impact the performance of the microscope in long-term use.

While the way that the present disclosure addresses the disadvantages of the prior art will be discussed in greater detail below, in general, the present disclosure provides a microscope disinfecting system comprising a pair of adapters, an electric connector configured to provide electric power for the activation of a light source configured within each of the pair of adapters, and a substantially Y-shaped connector that electrically connects the electric connector with the pair of adapters through a wiring unit.

In an embodiment, each of the adapters comprises an inner cavity dimensioned to receive an ocular lens of a microscope to disinfect the ocular lens of a microscope. The inner cavity comprises the light source configured to produce ultraviolet C (UV-C) light. The light source may be a light-emitting diode (LED) bulb capable of producing ultraviolet C (UV-C) light in the range of 200 nm-280 nm in wavelength.

In an embodiment, the electric connector is a power plug capable of drawing power from the external direct current (DC)/alternating current (AC) supply available in the vicinity of the microscope.

In an embodiment, the wiring unit comprises one or more components for an efficient electric power transfer consisting of a transformer, rectifier, filters and regulators, ferrite bead, and charger connector.

In an embodiment, the microscope disinfecting system further comprises a provision for housing at least one battery to provide electric power for the activation of the light source configured within each of the pair of adapters in the absence of the electric connector.

In an embodiment, each of the pair of adapters comprises a base primary ring connectable to an adapter ring. The base primary ring embodies the light source. The adapter ring is interchangeable and is meant to be replaced by a differently sized adapter ring for fitting a specific ocular size. The interchangeable adapter ring is at least sized as 35 mm, 40 mm, and 45 mm to fit a 50 mm base primary ring. The base primary ring and the adapter ring together fit snugly over the ocular lens of the microscope to direct the ultraviolet C (UV-C) light from the light source.

These and other features and advantages of the present invention will become apparent from the detailed description below, in light of the accompanying drawings.

### BRIEF DESCRIPTION OF DRAWINGS

A more complete understanding of the present disclosure may be derived by referring to the detailed description and claims when considered in connection with the figures, wherein like reference numerals refer to similar elements throughout the figures, and

FIG. 1 illustrates a front perspective view of a microscope disinfecting system, according to an embodiment of the present invention.

FIG. 2 illustrates another front-perspective view of the microscope disinfecting system of FIG. 1, wherein the electrical connector and the wiring unit are omitted for the sake of simplicity.

FIGS. 3 and 4 illustrate a perspective view of a variety of adapters of various sizes, according to an embodiment of the present invention.

FIG. 5 illustrates a front-perspective view of the microscope disinfecting system of FIG. 1 being used to disinfect a pair of ocular lenses of a microscope, according to an exemplary embodiment.

### DETAILED DESCRIPTION

The following description is of exemplary embodiments of the invention only and is not intended to limit the scope, applicability, or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms, and the like and still fall within the scope of the present invention. Thus, the detailed description herein is presented for purposes of illustration only and not for limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

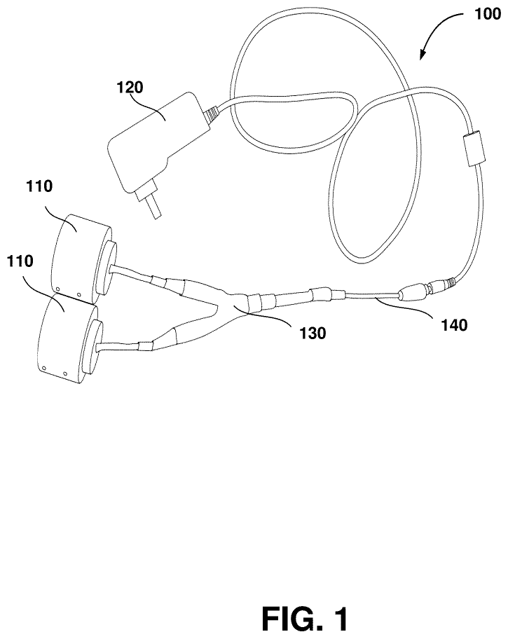

The microscope disinfecting system and possible embodiments will now be described with reference to the accompanying drawings, particularly FIGS. 1-5.

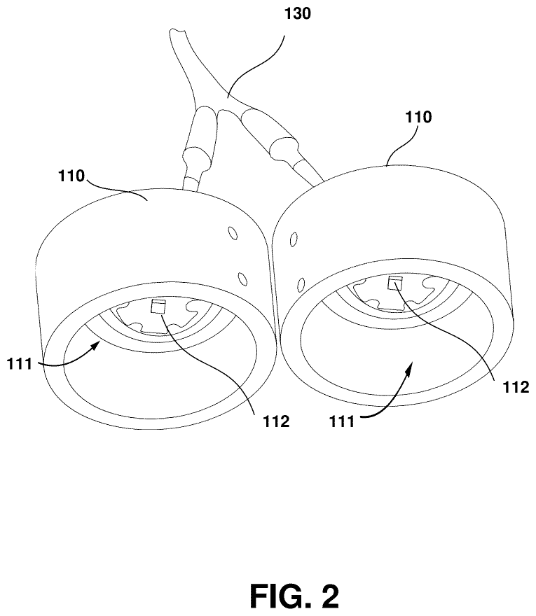

Reference is initially made to FIGS. 1-2 that illustrate front-perspective views of a microscope disinfecting system 100, according to an embodiment of the present invention. The microscope disinfecting system 100 includes at least one adapter 110, such as a pair of adapters 110 and an electric connector 120. Although the embodiment shows the presence of two independent adapters 110, it should be understood that instead of a pair of adapters, there may be a single adapter that would encompass ocular lens 210 of the microscope 200 (not seen). The number of adapters might depend on the number of lens 210 present in the microscope 200. A substantially Y-shaped connector 130 electrically connects the electric connector 120 with the pair of adapters 110 through a wiring unit 140.

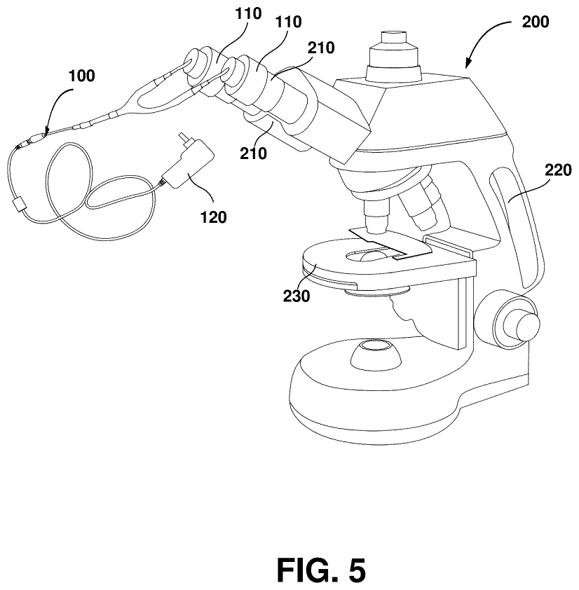

Each adapter 110 selected from the pair of adapters 110 is identical in design and shape and is collectively designated as “the pair of adapters 110” and individually designated as “the adapter 110”. Each adapter 110 is substantially cylindrical in shape and includes an inner cavity 111 that is dimensioned to receive an ocular lens 210 (FIG. 5) of a microscope 200 (FIG. 5). The adapter 110 snugly fits on the top of the ocular lens 210 (FIG. 5) of the microscope 200 (FIG. 5) to disinfect the ocular lens 210 (FIG. 5) of the microscope 200 (FIG. 5).

Referring to FIG. 2, the inner cavity 111 of the adapter 110 includes at least one light source 112. In an embodiment as seen in FIG. 2, the light source 112 is a light-emitting diode (LED) bulb 112 that is placed inside an inner cavity 111 of the adapter 110. The light-emitting diode (LED) bulb 112 is configured to produce ultraviolet C (UV-C) light (also referred as “Short-wave UV, germicidal UV, ionizing radiation at shorter wavelengths, completely absorbed by the ozone layer and atmosphere: hard UV”) that is in range of 200 nanometer (nm)-280 nanometer (nm) in wavelength and known to be ideal wavelength for killing microorganisms such as but not limited to: bacteria and fungus. UV-C light is germicidal, such that it deactivates the deoxyribonucleic acid (DNA) of microorganisms like bacteria, viruses, fungi, and other pathogens. The light-emitting diode (LED) bulb 112 is placed (snugly fit) over the ocular lens 210 (eyepiece) of the microscope 200 (FIG. 5) needing disinfection treatment. The ultraviolet C (UV-C) light shines through the ocular lens 210 (eyepiece) of the microscope 200 (FIG. 5), thereby killing the biological growth of the microorganisms such as bacteria and fungi.

In another embodiment (not shown in figures), the light source 112 is any other lighting unit that can effectively produce ultraviolet C (UV-C) light that is 200 nanometer (nm)-280 nanometers (nm) in wavelength and the lighting unit could include but not limited to: halogen bulb, sodium bulb, condensed fluorescent light (CFL) bulb, light amplification by stimulated emission of radiation (LASER), gas-discharge lamp, fluorescent lamp, tube light and so on.

The electric connector 120 is configured to provide electric power for the activation of the light source 112 of the pair of adapters 110. The electric connector 120 is configured to draw power from the external direct current (DC)/alternating current (AC) supply available in the vicinity of the microscope 200 (FIG. 5). In an embodiment as seen in FIG. 1, the electric connector 120 is a power plug configured to take electric power from a power socket of a laboratory in which the microscope 200 (FIG. 5) is placed. The wiring unit 140 could include various components necessary for the efficient electric power transfer, such as but not limited to: a transformer, rectifier, filters and regulators, ferrite bead, charger connector, and so on.

In another embodiment (not shown in figures), the microscope disinfecting system 100 may include provision for housing at least one battery/cell (not shown in figures) instead of just relying upon the electric connector 120. The battery/cell (not shown in figures) may be rechargeable/removable in use and may include, but not be limited to: lithium-ion battery, lead acid battery, graphite cell, alkaline battery, nickel-cadmium battery, and so on. The battery/cell is configured to provide electric power for the activation of the light source 112 of the pair of adapters 110. The advantage of the battery/cell compared to the electric connector 120 is that the microscope disinfecting system 100 involving the use of the battery/cell could be used in locations where electricity is not readily available.

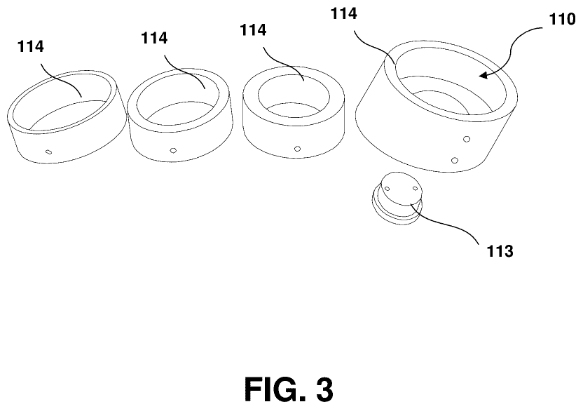

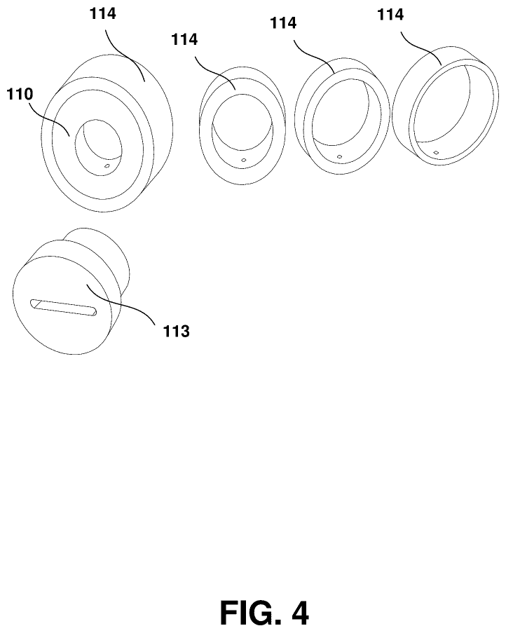

FIG. FIGS. 3 and 4 illustrate perspective views of a variety of adapters 110 of various sizes, according to an embodiment of the present invention. Each of the adapters 110 comprises a base primary ring 113 that may be connected to an adapter ring 114. The base primary ring 113 includes a light-emitting diode (LED) bulb 112. The adapter ring 114 is interchangeable and is meant to be removed or replaced by differently sized adapter ring 114 for fitting a specific ocular size. In various embodiments, as seen in FIGS. 3-4, the interchangeable adapter ring 114 may have sizes in the range of 35 mm, 40 mm, and 45 mm that can fit with the 50 mm base primary ring 113. However, it should be understood that the interchangeable adapter ring 114 and the base primary ring 113 can have any other sizes depending on the specifications of the microscope 200. The multiple sizes of the interchangeable adapter rings 114 allow the microscope disinfecting system 100 to work on the ocular lens 210 (eyepiece) of any size ranging between 30 and 50 millimeters (mm) of the microscope 200. The base primary ring 113 and the adapter ring 114 together fit snugly over the ocular lens 210 (eyepiece) of the microscope 200 and direct the ultraviolet C (UV-C) light coming from the light source 112 through the entire optical system inside both the binoculars and optical head of the microscope 200.

FIG. 5 illustrates a front-perspective view of a microscope disinfecting system 100 being used to disinfect a pair of ocular lenses 210 of a microscope 200. The microscope 200 is conventional in design and already known in the art. Although the microscope disinfecting system 100 is suitable for use with a variety of light microscopes, it is useful to review the basic microscope structure and function to appreciate the present invention. The microscope 200 broadly includes a stand 220 to which all the component pieces of the microscope 200 are mounted. In the embodiment shown, in FIG. 5, a viewing body is a binocular, comprising the body and a pair of ocular lenses 210 that are to be disinfected. Microscope 200 further comprises microscope stage 230, which is mounted to stand 220.

It should be understood that microscope disinfecting system 100 is suitable for use with a microscope configured with any type of viewing body such as but not limited to monocular, binocular, trinocular, video, etc. Further, it should be understood that the microscope disinfecting system 100 is useful in killing microorganisms such as but not limited to: fungi, bacteria, protozoa, algae, viruses, rickettsiae, etc, and other pathogens and so on of various optical elements of the microscope 200, wherein the various optical elements could include but not limited to: ocular lens (eyepieces), ports, microscope heads, eye tubes, objective lenses and so on.

In an embodiment, the microscope disinfecting system 100 is made of a bactericidal and/or fungicidal and or antimicrobial material/coating to further prevent the growth (spread) of the microorganisms, such as but not limited to: bacteria, fungi on the surface of the microscope disinfecting system 100. The bactericidal and/or fungicidal and or antimicrobial material/coating could include, but not be limited to copper, copper alloy surfaces, silver, organosilanes, nanomaterials such as titanium dioxide, zinc oxide, and other chemical substances having antimicrobial properties, and so on.

The various components and parts of the various embodiments of the microscope disinfecting system 100 of the present invention are similar and interchangeable. Further, it should be understood that the components of the microscope disinfecting system 100 can be made of any material and any size depending on the type of microscope 200. Further, the shape of the microscope disinfecting system 100 could be modified depending on the requirements of the microscope 200.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

What is claimed is:

1. A microscope disinfecting system (100), comprising:
at least one adapter (110);
an electric connector (120) configured to provide electric power for the activation of a light source (112) configured within the at least one adapter (110); and
wherein the at least one adapter (110) comprises an inner cavity (111) dimensioned to receive at least one ocular lens (210) of a microscope (200) to disinfect the ocular lens (210) of a microscope (200).

2. The microscope disinfecting system (100) of claim 1, wherein the at least one adapter (110) comprises a pair of adapters (110).

3. The microscope disinfecting system (100) of claim 1 further comprising a substantially Y-shaped connector (130) that electrically connects the electric connector (120) with the at least one adapter (110) through a wiring unit (140).

4. The microscope disinfecting system (100) of claim 1, wherein the inner cavity (111) comprises the light source (112) configured to produce ultraviolet C (UV-C) light.

5. The microscope disinfecting system (100) of claim 1, wherein the light source (112) is a light-emitting diode (LED) bulb (112) capable of producing ultraviolet C (UV-C) light in the range of 200 nm-280 nm in wavelength.

6. The microscope disinfecting system (100) of claim 1, wherein the electric connector (120) is a power plug capable of drawing power from the external direct current (DC)/alternating current (AC) supply available in the vicinity of the microscope (200).

7. The microscope disinfecting system (100) of claim 3, wherein the wiring unit (140) comprises one or more components for an efficient electric power transfer consisting of a transformer, rectifier, filters and regulators, ferrite bead, and charger connector.

8. The microscope disinfecting system (100) of claim 1 further comprising a provision for housing at least one battery to provide electric power for the activation of the light source (112) configured within each of the pair of adapters (110) in the absence of the electric connector (120).

9. The microscope disinfecting system (100) of claim 2, wherein each of the pair of adapters (110) comprises a base primary ring (113) connectable to an adapter ring (114).

10. The microscope disinfecting system (100) of claim 9, wherein the base primary ring (113) embodies the light source (112).

11. The microscope disinfecting system (100) of claim 9, wherein the adapter ring (114) is interchangeable and is meant to be replaced by a differently sized adapter ring (114) for fitting a specific ocular size.

12. The microscope disinfecting system (100) of claim 11, wherein the interchangeable adapter ring (114) is at least sized as 35 mm, 40 mm, and 45 mm to fit a 50 mm base primary ring (113).

13. The microscope disinfecting system (100) of claim 9, wherein the base primary ring (113) and the adapter ring (114) together fit snugly over the ocular lens (210) of the microscope (200) to direct the ultraviolet C (UV-C) light from the light source (112).

14. A microscope disinfecting system (100), comprising:
at least one adapter (110), wherein each adapter (110) comprises an inner cavity (111) dimensioned to receive an ocular lens (210) of a microscope (200); and
an electric connector (120) configured to provide electric power for the activation of a light source (112) configured within each of the pair of adapters (110) to disinfect the ocular lens (210) of a microscope (200).

15. The microscope disinfecting system (100) of claim 14 further comprising a substantially Y-shaped connector (130) that electrically connects the electric connector (120) with the at least one adapter (110) through a wiring unit (140).

16. The microscope disinfecting system (100) of claim 15, wherein the at least one adapter (110) comprises a base primary ring (113) connectable to an adapter ring (114).

17. The microscope disinfecting system (100) of claim 16, wherein the base primary ring (113) embodies the light source (112).

18. The microscope disinfecting system (100) of claim 17, wherein the light source (112) is a light-emitting diode (LED) bulb (112) capable of producing ultraviolet C (UV-C) light in the range of 200 nm-280 nm in wavelength.

19. The microscope disinfecting system (100) of claim 15, wherein the wiring unit (140) comprises one or more components for an efficient electric power transfer consisting of a transformer, rectifier, filters and regulators, ferrite bead, and charger connector.
